(19) United States
(12) Patent Application Publication
(10) Pub. No.: US 2024/0170128 A1
(43) Pub. Date: May 23, 2024

(54) SYSTEM FOR MANAGING AND TRACKING FOOD INTAKE

(71) Applicants: Michel CARON, Ottawa, CA (US); Kevin A. STAAB, Harrison, OH (US)

(72) Inventors: Michel CARON, Ottawa, CA (US); Kevin A. STAAB, Harrison, OH (US)

(21) Appl. No.: 18/058,477

(22) Filed: Nov. 23, 2022

(51) Int. Cl. G16H 20/60; A47G 19/02; G06V 20/68

(52) U.S. Cl. CPC G16H 20/60; A47G 19/025; G06V 20/68

(57) ABSTRACT

In one embodiment, a system for aiding a user to control nutritional intake includes a portions management (PM) plate having a base with one or more compartments each having an open top, and a cover having one or more cutouts each corresponding to an open top of a compartment of the base, the cover being rotatably mounted relative to the base to varying degrees of alignment of the cutouts to the corresponding open tops of the compartments to thereby selectively occlude or expose the open tops and control the amount of food that can be inserted into the one or more compartments for support on a platter over which the portions management plate is disposed. An app for use with a smart phone or the like can also be included and used in conjunction with the physical PM plate, or with a virtual PM that can be displayed by the app.

### TECHNICAL FIELD

The present disclosure relates generally to dieting and weight control.

### BACKGROUND

Dieting and weight control are a societal concern and a multi-billion dollar industry. To the individual, they have profound mental and physical health implications. With varying effectiveness, many expedients are available to assist dieters manage the quality and quantity of their food intake and caloric consumption. Some of these relate to meal portion control, while others relate to meal scheduling, exercise, or myriad other factors or combinations of factors that impact an individual's weight and physical and mental health, whether for adults or children. Prevalent in the art are references generally directed to plates/food trays for infants including portion size compartments. Also common are food weighing systems that are integral to a plate. In addition, food trays that have compartments sized to control portion intake of unhealthy foods (e.g., a protein compartment that is much larger than a dessert compartment) are known.

The advent of smart phones and mobile devices has armed individuals with powerful tools to assist in their dieting endeavors. “Apps” for tracking and counting calories, scheduling meals, managing fasting regimens, and so on, are ubiquitous.

### OVERVIEW

Described herein is a system for aiding a user to control nutritional intake includes a portions management (PM) plate having a base with one or more compartments each having an open top, and a cover having one or more cutouts each corresponding to an open top of a compartment of the base, the cover being rotatably mounted relative to the base to varying degrees of alignment of the cutouts to the corresponding open tops of the compartments to thereby selectively occlude or expose the open tops and control the amount of food that can be inserted into the one or more compartments for support on a platter over which the portions management plate is disposed. An app for use with a smart phone or the like can also be included and used in conjunction with the physical PM plate, or with a virtual PM that can be displayed by the app.

### BRIEF DESCRIPTION OF THE DRAWINGS

The accompanying drawings, which are incorporated into and constitute a part of this specification, illustrate one or more examples of embodiments and, together with the description of example embodiments, serve to explain the principles and implementations of the embodiments.

In the drawings:

FIG. 1 is a schematic view of a dieting system in accordance with certain embodiments;

FIG. 2 is a block diagram of a mobile device for executing an app for assisting dieters to manage their food intake in accordance with certain embodiments;

FIG. 3A is a flow diagram of some steps that may be facilitated by the app in the initial set up phase accordance with certain embodiments;

FIG. 3B is a flow diagram of some steps that may be facilitated by the app during actual use for aid in controlling a user's nutritional intake in accordance with certain embodiments;

FIG. 3C is a view of a displayed captured food image or video and includes an overlaid interactive graphical image of a virtual PM plate with a virtual cover in accordance with certain embodiments;

FIG. 4 is a block diagram of an app having various modules that may be locally stored and/or executed on a mobile device and used in conjunction with or in lieu of the PM plate in accordance with certain embodiments; and

FIG. 5 is a block diagram that illustrates one example of a computer system that can be employed to execute one or more embodiments of the present disclosure.

### DESCRIPTION OF EXAMPLE EMBODIMENTS

Example embodiments are described herein in the context of a System for Managing and Tracking Food Intake. The following description is illustrative only and is not intended to be in any way limiting. Other embodiments will readily suggest themselves to those of ordinary skill in the art having the benefit of this disclosure. Reference will be made in detail to implementations of the example embodiments as illustrated in the accompanying drawings. The same reference indicators will be used to the extent possible throughout the drawings and the following description to refer to the same or like items.

In the description of example embodiments that follows, references to “one embodiment”, “an embodiment”, “an example embodiment”, “certain embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. The term “exemplary” when used herein means “serving as an example, instance or illustration.” Any embodiment described herein as “exemplary” is not necessarily to be construed as preferred or advantageous over other embodiments.

In the interest of clarity, not all of the routine features of the implementations described herein are shown and described. It will be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

In accordance with this disclosure, the components, process steps, and/or data structures described herein may be implemented using various types of operating systems, computing platforms, computer programs, and/or general purpose machines. Devices of a less general purpose nature, such as hardwired devices, field programmable gate arrays (FPGAs), application specific integrated circuits (ASICs), or the like, may also be used without departing from the scope and spirit of the inventive concepts disclosed herein. Where a method comprising a series of process steps is implemented by a computer or a machine and those process steps can be stored as a series of instructions readable by the machine, they may be stored on a tangible medium such as a computer memory device (e.g., ROM (Read Only Memory), PROM (Programmable Read Only Memory), EEPROM (Electrically Erasable Programmable Read Only Memory), FLASH Memory, Jump Drive, and the like), magnetic storage medium (e.g., tape, magnetic disk drive, and the like), optical storage medium (e.g., CD-ROM, DVD-ROM, paper card, paper tape and the like) and other types of program memory.

Herein, reference to a computer-readable or machine-readable storage medium encompasses one or more non-transitory, tangible storage media possessing structure. As an example and not by way of limitation, a computer-readable storage medium may include a semiconductor-based circuit or device or other IC (such, as for example, a field-programmable gate array (FPGA) or an ASIC), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate. Herein, reference to a computer-readable storage medium excludes any medium that is not eligible for patent protection under 35 U.S.C. § 101. Herein, reference to a computer-readable storage medium excludes transitory forms of signal transmission (such as a propagating electrical or electromagnetic signal per se) to the extent that they are not eligible for patent protection under 35 U.S.C. § 101. A computer-readable non-transitory storage medium may be volatile, nonvolatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

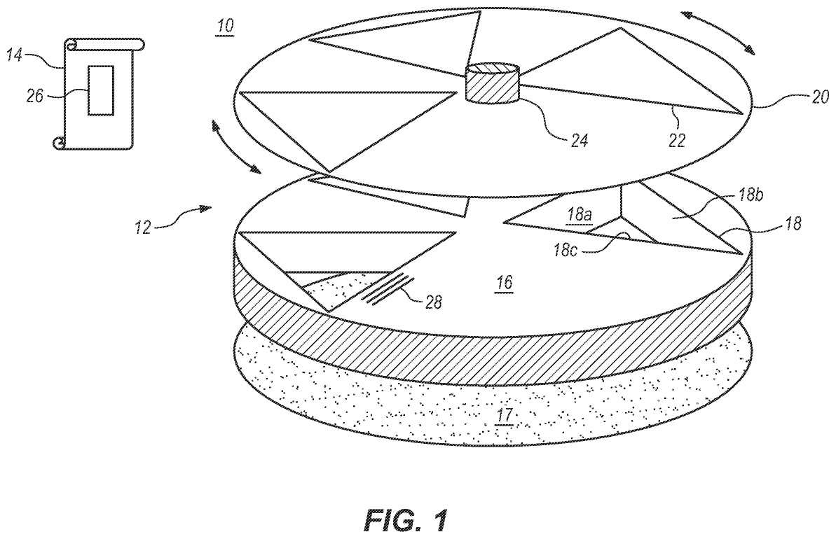

FIG. 1 is a schematic view of a dieting system 10 in accordance with certain embodiments. Generally, system 10 includes an adjustable portions management (PM) plate 12, and an information library 14, such as a physical sheet or booklet or an electronic database. PM plate 12 is operable to measure out a prescribed amount of food to be placed on a platter 17 in consultation with information obtained from library 14 as detailed below, enabling a user to carefully control his/her food intake in a meal.

PM plate 12 comprises a base 16 having one or more compartments 18 that have sides 18a but are open at their top 18b and bottom 18c. The compartments 18 are shown to be pie-shaped, but other shapes are contemplated. PM plate 12 also comprises a cover 20 rotatably attached to base 16 and having cutouts 22 that are adjustably alignable with top openings 18b of the compartments 18 in the base. PM plate 12 is operable for placement atop platter 17 to measure out portions of food to be served on the platter. A dial 24 integrally formed with cover 20 serves to rotate the cover to change the alignment of the cutouts 22 with the top openings 18b of the corresponding compartments 18 and thereby adjust the amount of food that can be placed in the platter 17 by way of the compartments. Cover 20 is normally rotatably attached to base 16, but can be removed for easy cleaning. The base 16 and cover 20 are made from food-safe materials such as certain plastics, alloys, and the like.

In certain embodiments, the number of compartments 18 and corresponding cutouts 22 in cover 20 is three. Each of these three in turn can correspond to a type of food and may be labelled accordingly. In such a three-type example, these are proteins (for example, red meat, poultry, fish, nuts); greens (for example lettuce, kale, broccoli and other vegetables and even fruits); and grains (for example cereal, bread). By rotating the dial 24, the user is able to control the capacity of the compartments 18 to hold these types of food, and commensurately, the total capacity of the PM plate 12 for a particular meal.

In one example, system 10 can be used by a dieter to lose weight. Specifically, a healthy adult male wishing to reduce his weight from 200 pounds to 180 pounds over a span of 3 months can use the adjustable PM plate 12 in conjunction with a cover table 26 in library 14 to achieve this goal. Cover table 26, which can be in printed or electronic form, relates various dieting parameters to corresponding settings of the PM plate 12 as implemented by positioning of the cutouts 22 of cover 20 over the corresponding compartments 18 and thereby controlling food portion size. For example, it can inform the dieter of the number of meals per day they should eat for each day of the week, and also the position to which to turn dial 24 to adjust the portion size of each type of food in each of these meals. If a more aggressive diet regimen is desired—for example the same weight loss in two months rather than three—the information in table 26, including the settings of dial 24, can be different. For example, for one or more meals of at least one of the days of the week, the more aggressive regimen can require presetting the dial position for half open compartments 18 rather than three quarters open.

In addition to weight, dieting parameters in table 26 can also relate to waist size or other body dimensions, body mass index, gender, age, height, general health, fitness, activeness of lifestyle, among many other factors. The dieter can select the set of parameters that best characterize him and their goals, and this set is correlated in the table 26 to a particular setting of dial 24, which in turn determines portion size per meal. A calorie intake indication can also be provided to the dieter by the table 26—for example, the dieter can be informed by the table that an intake of 2,500 calories per day needs to be observed to meet the dieting goal of loss of 20 pounds in three months in the above example.

In one example, indicia 28 can be provided in PM plate 12 that, together with the location of an edge of a cutout 22, indicate the capacity of the compartments 18 based on the extent of alignment of their openings 18b with the cutouts 22. The indicia can be labeled in fractional units—fully open, half open, three quarters open—and/or in absolute units-6 ounces, 3 ounces, 1.5 ounces, etc. Other fractions and amounts are of course contemplated. The indicia 28 can also be labeled to correspond to information in the table 26 such as user weight loss goal, calories, etc. For example, for the above desired regimen of a reduction of weight to 180 pounds by an adult male, the table 26 can indicate that the cover 20 should be placed at a 180 lb setting, as indicated by indicia 28, for each of three daily meals.

Advantages of the system 100 include:
Ensuring the dieter is eating the proper portion size for his/her desired weight goal
Achieving and maintaining weight loss goals faster
Eliminating calorie and points counting
Avoiding over-eating

System 10 thus uses preset portion sizes for protein, carbohydrates, and greens according to the dieter's goal weight. The dieter selects his/her goal weight, serves pre-set portions of his/her favorite foods in the PM plate 12, and uses the plate for the prescribed number of meals per day. There is no counting calories, measuring food, or counting points, and results are achieved as quickly and efficiently as possible. The results are tailored to the dieter's needs, and are realized using the PM plate 12 in consultation with cover table 26, whose information contains scientifically researched portion sizes for protein, carbs, and vegetables, tailored individual's physiology according to gender, age, and so on, so that the dieter can eat the foods they enjoy and successfully lose weight. The settings and information stored in cover table 26 can thus be derived from expert recommendations such as those from dieticians, nutritionists, medical personnel, and nutritional and medical studies on the subject.

As mentioned above, cover table 26 can be in either printed or electronic form. In the latter case, it can be retained in an “app” (application) of a mobile device operable to assist dieters to manage their food intake to for example lose weight or maintain a current weight, or even to gain weight, or otherwise achieve any of myriad health benefits that inure from proper food intake control and management.

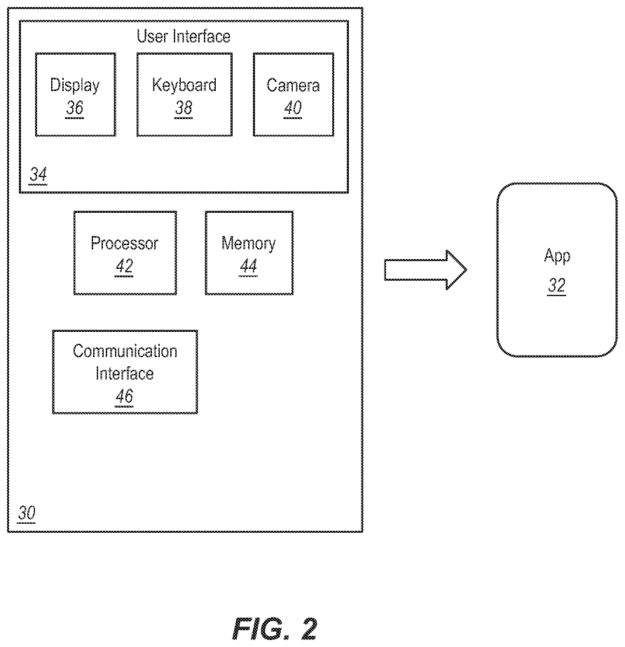

FIG. 2 shows in block diagram a mobile device 30 for executing an app 32 for assisting dieters to manage their food intake in accordance with certain embodiments. The app 32 can be part of system 10 above, which includes PM plate 12 and information library 14 with cover table 26, in this case in electronic form. Components of the app 32 are described in more detail below. In some embodiments, the app 32 can be stand-alone, without the PM plate 12, and instead generating a virtual plate 12′ for implementing a dieting regimen as detailed below.

Mobile device 30 comprises a user interface 34 having a display 36, keyboard 38, and camera 40. Mobile device 30 also comprises a processor 42 for executing code and other instructions, and a memory 44 for storing said code, along with an operating system and other code and information, a browser, and so on, and a communication interface 46 for effecting wired or wireless communication with other devices over cellular, Wifi, LAN networks and the like.

App 32 may be downloadable from an online app store in a conventional manner, and comprises programming code that is at least partially stored in memory 44 and executed by processor 42. In certain embodiments, some of the code is also stored and executed remotely from mobile device 30, for example in cloud devices such as servers that are accessible through wired or wireless networks that may include the Internet.

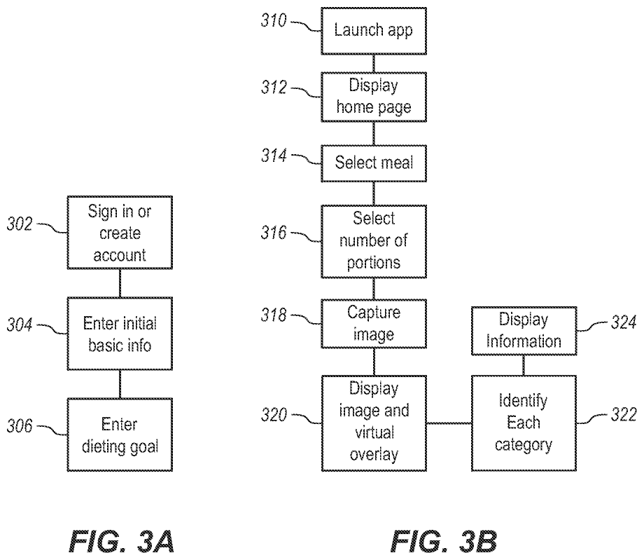

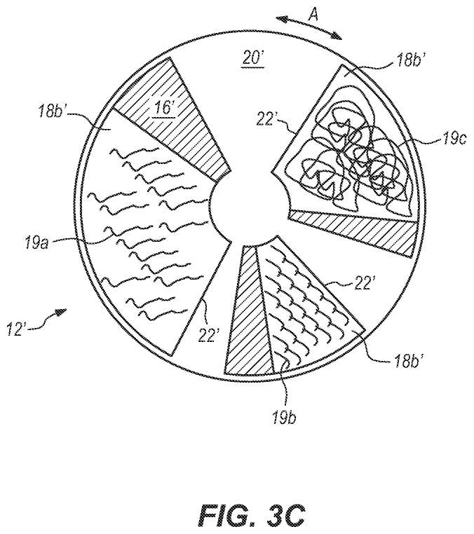

FIG. 3A is a flow diagram of some steps that may be facilitated by the app 32 in the initial set up phase accordance with certain embodiments. The ordering of steps, it will be appreciated, may be different than that presented and shown. At step 302, the user is prompted to sign in to their account or create an account. If the user is new, then initial basic information is entered at 304, such as name, age, gender, location, and, in addition, physiological information relating to for example weight, height, waist size, BMI (body mass index), unit preference (metric, imperial), and so on. At 306, a dieting goal is entered, such as reduction to a certain goal weight, realization of a certain BMI or waist size, weight maintenance, and so on, dieting period (30 days, 6 months, etc.). Based on the initial basic information and the dieting goal, a dieting regimen is created by the app 32, in accordance with expert information such as derived from dieticians, nutritionists, medical personnel, nutritional and medical studies on the subject, and other authoritative sources. Such information may be digitally stored by app 32, in a version of cover table 26 in library 14. In certain embodiments, the dieting regimen specifies the total daily calorie intake permitted, for example 2,500 calories/day, and informs the dieter of this amount.

FIG. 3B is a flow diagram of some steps that may be facilitated by the app 32 during actual use for aid in controlling a user's nutritional intake. The ordering of steps, it will be appreciated, may be different than that presented and shown. When the user is ready to prepare a meal to eat, the user launches the app at step 310, and a home page of the app is displayed at 312. The home page may contain information such as the total daily calorie intake permitted. It may also contain the date, and selectable icons representing meals such as breakfast, lunch, dinner, and snack. From these, the user is prompted at 314 to select the meal at hand—for example breakfast. The user is then queried about the number of portions in the breakfast at 316, and which of the three categories—carb, greens, or proteins in this example—each portion is. The user is then prompted to take an overhead photograph or video of the meal at 318. To do this, the app 32 activates a camera of the mobile device, displaying the view of the platter 17 and supported meal, with a button that the user can select to capture a photograph or a live video of that view. In certain embodiments, the capture of the still or video image is launched automatically by the app 32. At 320, the captured food image or video is displayed to the user, and as seen in FIG. 3C, includes an overlaid interactive graphical image of a virtual PM plate 12′ with a virtual cover 20′ that can be rotated by the user in the directions of arrow A in the still photo, or in the live video in real time as the meal is being filmed. Virtual cover 20′ is fully or partially opaque, concealing the food in the image it overlays, except at compartment openings 18b′ through which the food 19a, 19b, 19c (collectively “19”) in the captured image or video is visible. The displayed compartment openings 18b′ are adjustable in size as a function of the displayed degree of rotation of the virtual cover 20′ relative to the base 16′, which offsets the alignment of the cutouts 22′ with the compartment openings 18b′ and thereby reduces the size of the openings in the image/video. The amount of reduction, if any, and hence the displayed area of the compartment openings 18b′ in which food is visible, may be determined by the app 32 in consultation with its digitally stored version of cover table 26 in library 14, analogous to the manner of the physical PM plate 12 described above in which the user rotates the physical cover 20 to control the overlap and offset of the cutouts 22 and compartment tops 18b of PM plate 12. In certain embodiments, the overlaid image of virtual PM plate 21′, and the virtual cover 20′ in particular, is interactive, permitting the user to rotate the virtual cover as shown by arrow A for example by an appropriate rotating or moving gesture of a finger on the touchscreen of the mobile device. Alternatively, the amount of offset is automatically determined by the app 32 based on the desired diet parameters as described above. In either case, a limited amount of food 19 in each category is shown through the displayed virtual PM plate 12′, thereby providing a guide to the user of the amount of said food to be served and consumed for that meal. The user can adjust the actual portions on the serving platter to match those displayed in the virtual PM plate 12′ through the displayed adjustable compartment openings 18b′ by removing excess food or adding more as needed. In certain embodiments, the physical PM plate 12 can also be used, in conjunction with the virtual plate 12′, with the virtual plate serving to guide the rotation of the physical cover 20 of the physical PM plate through the varying degrees of alignment of the cutouts 22 to the corresponding open tops 18b of the compartments 18 to thereby selectively occlude or expose the open tops and control the amount of food that can be inserted into the one or more compartments and supported on the platter 17 over which the PM plate 12 is disposed.

At 322, the user is prompted to identify the actual food 19 displayed in each of the compartment openings 18′. For example, if carbs was selected, the user is prompted to identify the carbs as whole wheat bread, potatoes, rice, etc; if proteins were selected, salmon, beef, lamb, etc., may be identified; and if a greens were selected, kale, lettuce, squash, etc., may be identified. From these identified foods, the app 32 provides a number (for example “3.2 ounces”) indicative of the amount of food recommended for each category. In certain embodiments, a manual override by the user can be permitted so that the user can adjust and enter his/her own numbers, in weight or calories, for instance. In certain embodiments, it also determines the total amount of calories in the meal, and at 324, displays this total, along with other pertinent information such as the total calorie count of all the meals that have been tracked that day, that week, or for the entire regimen. It may also display the number of calories remaining to the user for that day, after subtracting the total meals from the permitted amount for the day.

In certain embodiments, the app 32 can also include a food diary to assist the user to track his/her food intake over a period of time. Water/liquid intake can be tracked in a similar manner.

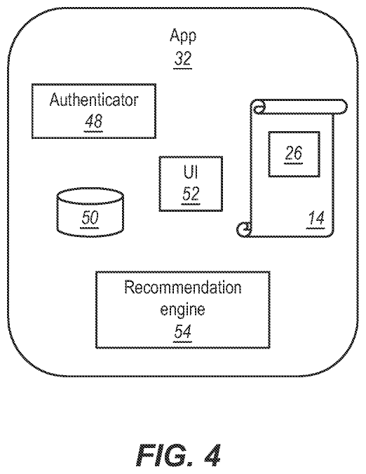

As seen in the block diagram in FIG. 4, app 32 contains various modules that may be locally stored and/or executed, on the mobile device 30; or they may be distributed across the cloud for storage and execution on remote devices; or a combination of local and remote storage and execution may be employed.

In certain embodiments, the various modules are operable to carry out the functions and steps described above, and can include an authenticator 44 for authenticating the user as a valid holder of an account registered with the app, or prompting the user to create such an account; a library 14 with table 26 as described above; an information storage component 50 for storing user information, including account information, device information, and user current physical information and dieting goals (e.g. weight, BMI, waist size, age, height, etc, as described above), and in some embodiments, the library 14 and table 26; a user interface component 52 for facilitating interaction with the user such as entry of information by the user and presentation of information to the user including the overlaid PM plate 12′; and a recommendation engine 54 for determining caloric and weight recommendations for meals and food categories in consultation with table 26 as described above.

In certain embodiments, the term “app” (such as application 32) refers to a desktop or laptop computer device application, mobile device application, web application, browser, or browser extension. For example, application 32 may be configured as an application installed on computing device or may be a web application operated through a browser or browser extension on a computing device or a mobile phone application executing on an IOS or Android operating system.

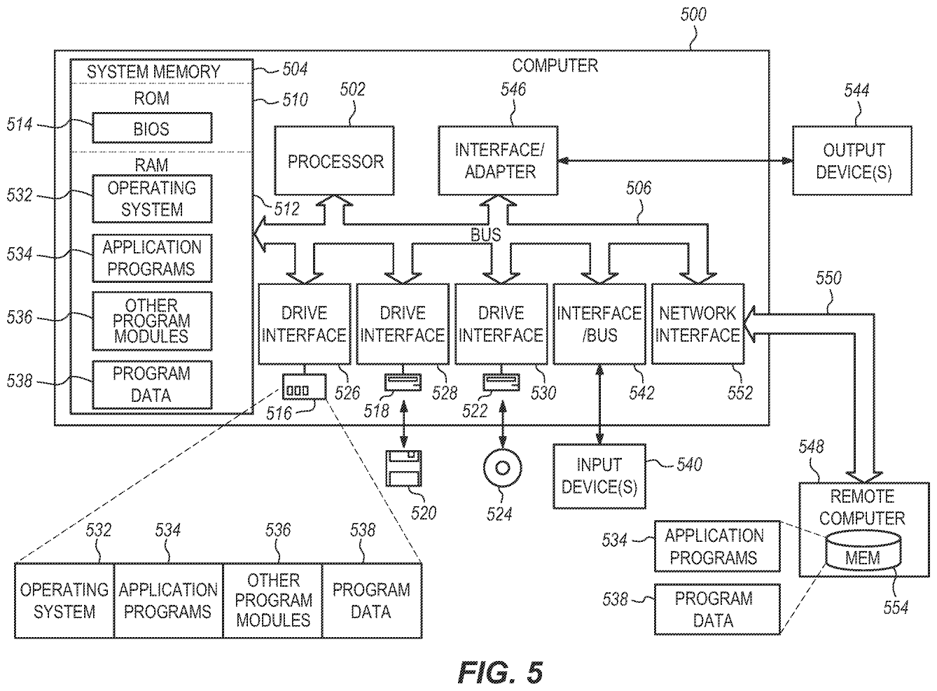

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software and hardware, such as shown and described with respect to the computer system of FIG. 5. Furthermore, portions of the embodiments may be a computer program product on a computer-usable storage medium having computer readable program code on the medium. Any non-transitory, tangible storage media possessing structure may be utilized including, but not limited to, static and dynamic storage devices, hard disks, optical storage devices, and magnetic storage devices, but excludes any medium that is not eligible for patent protection under 35 U.S.C. § 101 (such as a propagating electrical or electromagnetic signals per se). As an example and not by way of limitation, computer-readable storage media may include a semiconductor-based circuit or device or other IC (such, as for example, a field-programmable gate array (FPGA) or an ASIC), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate. A computer-readable non-transitory storage medium may be volatile, nonvolatile, or a combination of volatile and non-volatile, as appropriate.

Certain embodiments have also been described herein with reference to block illustrations of methods, systems, and computer program products. It will be understood that blocks and/or combinations of blocks in the illustrations, as well as methods or steps or acts or processes described herein, can be implemented by a computer program comprising a routine of set instructions stored in a machine-readable storage medium as described herein. These instructions may be provided to one or more processors of a general purpose computer, special purpose computer, or other programmable data processing apparatus (or a combination of devices and circuits) to produce a machine, such that the instructions of the machine, when executed by the processor, implement the functions specified in the block or blocks, or in the acts, steps, methods and processes described herein.

These processor-executable instructions may also be stored in computer-readable memory that can direct a computer or other programmable data processing apparatus to function in a particular manner, such that the instructions stored in the computer-readable memory result in an article of manufacture including instructions which implement the function specified. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

In this regard, FIG. 5 illustrates one example of a computer system 500 that can be employed to execute one or more embodiments of the present disclosure. Computer system 500 can be implemented on one or more general purpose networked computer systems, embedded computer systems, routers, switches, server devices, client devices, various intermediate devices/nodes or standalone computer systems. Additionally, computer system 500 can be implemented on various mobile clients such as, for example, a personal digital assistant (PDA), smart phone, laptop computer, pager, and the like, provided it includes sufficient processing capabilities.

Computer system 500 includes processing unit 502, system memory 504, and system bus 506 that couples various system components, including the system memory 504, to processing unit 502. Dual microprocessors and other multi-processor architectures also can be used as processing unit 502. System bus 506 may be any of several types of bus structure including a memory bus or memory controller, a peripheral bus, and a local bus using any of a variety of bus architectures. System memory 504 includes read only memory (ROM) 510 and random access memory (RAM) 512. A basic input/output system (BIOS) 514 can reside in ROM 510 containing the basic routines that help to transfer information among elements within computer system 500.

Computer system 500 can include a hard disk drive 516, magnetic disk drive 518, e.g., to read from or write to removable disk 520, and an optical disk drive 522, e.g., for reading CD-ROM disk 524 or to read from or write to other optical media. Hard disk drive 516, magnetic disk drive 518, and optical disk drive 522 are connected to system bus 506 by a hard disk drive interface 526, a magnetic disk drive interface 528, and an optical drive interface 530, respectively. The drives and associated computer-readable media provide nonvolatile storage of data, data structures, and computer-executable instructions for computer system 500. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and described herein.

A number of program modules may be stored in drives and RAM 510, including operating system 532, one or more application programs 534, other program modules 536, and program data 538. In some examples, the application programs 534 can include app 32, and the program data 538 can include the library 14 and table 26. The application programs 534 and program data 538 can include functions and methods programmed to implement the app 32, and particularly the method steps 302-306 and 310-324, such as shown and described herein.

A user may enter commands and information into computer system 500 through one or more input devices 540, such as a pointing device (e.g., a mouse, touch screen), keyboard, microphone, joystick, game pad, scanner, and the like. For instance, the user can employ input device 540 to edit or modify personal information and the like, or to move or rotate virtual cover 20′, and the like. These and other input devices 540 are often connected to processing unit 502 through a corresponding port interface 542 that is coupled to the system bus, but may be connected by other interfaces, such as a parallel port, serial port, or universal serial bus (USB). One or more output devices 544 (e.g., display, a monitor, printer, projector, or other type of displaying device) is also connected to system bus 506 via interface 546, such as a video adapter.

Computer system 500 may operate in a networked environment using logical connections to one or more remote computers, such as remote computer 548. Remote computer 548 may be a workstation, computer system, router, peer device, or other common network node, and typically includes many or all the elements described relative to computer system 500. The logical connections, schematically indicated at 550, can include a local area network (LAN) and/or a wide area network (WAN), or a combination of these, and can be in a cloud-type architecture, for example configured as private clouds, public clouds, hybrid clouds, and multi-clouds. When used in a LAN networking environment, computer system 500 can be connected to the local network through a network interface or adapter 552. When used in a WAN networking environment, computer system 500 can include a modem, or can be connected to a communications server on the LAN. The modem, which may be internal or external, can be connected to system bus 506 via an appropriate port interface. In a networked environment, application programs 534 or program data 538 depicted relative to computer system 300, or portions thereof, may be stored in a remote memory storage device 554.

While embodiments and applications have been shown and described, it would be apparent to those skilled in the art having the benefit of this disclosure that many more modifications than mentioned above are possible without departing from the inventive concepts disclosed herein. The invention, therefore, is not to be restricted based on the foregoing description. This disclosure encompasses all changes, substitutions, variations, alterations, and modifications to the example embodiments herein that a person having ordinary skill in the art would comprehend. Similarly, where appropriate, the appended claims encompass all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

What is claimed is:

1. A system comprising:
a portions management (PM) plate including:
a base having one or more compartments each having an open top, and
a cover having one or more cutouts each corresponding to an open top of a compartment of the base, the cover being rotatably mounted relative to the base to varying degrees of alignment of the cutouts to the corresponding open tops of the compartments to thereby selectively occlude or expose the open tops and control the amount of food that can be inserted into the one or more compartments for support on a platter over which the portions management plate is disposed.

2. The system of claim 1, wherein the plate includes indicia corresponding to the varying degrees of alignment, the indicia associated with different dieting regimens.

3. The system of claim 1, further comprising an information library usable in conjunction with the portions management plate and containing information for guiding a user through the varying degrees of alignment.

4. The system of claim 3, wherein the information is associated with dieting regimens and the varying degrees of alignment correspond to recommended caloric intakes associated with different dieting regimens.

5. The system of claim 3, further comprising an app for electronically managing the information library.

6. The system of claim 5, wherein the app is configured to capture an image or video of food on a platter and to indicate a degree of alignment of the cutouts to the corresponding open tops of the compartments.

7. The system of claim 6, wherein the degree of alignment is a function of a diet regimen selectable by the user in the app.

8. The system of claim 6, wherein the degree of alignment is a function of a meal selected from breakfast, lunch or dinner.

9. The system of claim 6, wherein the degree of alignment is a function of food type.

10. A machine-readable storage medium having stored thereon a computer program for aiding a user to control nutritional intake, the computer program comprising a routine of set instructions for causing the machine to perform the steps of:
receiving user physiological information;
receiving user dieting goals;
capturing an image of a user meal; and
displaying the captured image with an overlaid image of a virtual portions management (PM) plate, the overlaid image including a virtual cover having openings through which food from the captured image is displayed, the openings being adjustable in size as a function of the user dieting goals.

11. The machine-readable storage medium of claim 10, the set of instructions further causing the machine to perform the steps of:
prompting the user to select a meal associated with the captured image.

12. The machine-readable storage medium of claim 10, the set of instructions further causing the machine to perform the steps of:
prompting the user to select the number of portions in the captured image.

13. The machine-readable storage medium of claim 12, the set of instructions further causing the machine to perform the steps of:
prompting the user to identify the type of food in each portion.

14. The machine-readable storage medium of claim 10, wherein the physiological information relates to one or more of weight, height, waist size, BMI (body mass index), unit preference.

15. The machine-readable storage medium of claim 10, wherein the user dieting goals are selected from one or more of reduction to a certain goal weight, realization of a certain BMI or waist size, weight maintenance, and dieting period.
